(19) United States
(12) Patent Application Publication
(10) Pub. No.: US 2024/0392388 A1
(43) Pub. Date: Nov. 28, 2024

(54) DNA-METHYLATION-BASED CERTIFICATION OF QUALITY

(71) Applicants: Evonik Operations GmbH, Essen (DE); DEUTSCHES KREBSFORSCHUNGSZENTRUM STIFTUNG DES ÖFFENTLICHEN RECHTS, Heidelberg (DE)

(72) Inventors: Florian BÖHL, Neckargemund (DE); Frank LYKO, Hirschberg an der Bergstrabe (DE); Sanjanaa NAGARAJAN, Singapore (SG); Suki ROY, Singapore (DE); Sina LYKO-TÖNGES, Hirschberg an der Bergstrabe (DE); Geetha VENKATESH, Walzbachtal (DE); Walter PFEFFERLE, Langgöns (DE)

(73) Assignees: Evonik Operations GmbH, Essen (DE); DEUTSCHES KREBSFORSCHUNGSZENTRUM STIFTUNG DES ÖFFENTLICHEN RECHTS, Heidelberg (DE)

(21) Appl. No.: 18/689,298

(22) PCT Filed: Sep. 2, 2022
(86) PCT No.: PCT/EP2022/074412

§ 371 Date: Mar. 5, 2024

(30) Foreign Application Priority Data
Sep. 6, 2021 (EP) 21194988.8

(51) Int. Cl. C12Q 1/6888

(52) U.S. Cl. CPC C12Q 1/6888; C12Q 2600/124; C12Q 2600/154

(57) ABSTRACT

The present invention relates to a method of determining the supplier from which a test animal-derived product sample originates, the method comprising the steps of:
(a) determining a test methylation profile of one or more pre-selected methylation sites within the genomic material contained in the test animal-derived product sample; and
(b) comparing the test methylation profile determined in (a) with a panel of predetermined reference methylation profiles of the same biological taxon of the test animal from which the product sample derives, wherein each of the predetermined reference methylation profiles is from a different reference animal and/or different supplier,
   wherein if the test methylation profile of (a) is significantly similar to one of the predetermined reference methylation profiles, the test animal-derived product sample is confirmed of originating from a first supplier from which a first reference animal with the predetermined reference profile is obtained.

### FIELD OF THE INVENTION

The present invention relates to a method of determining certification of quality or source of origin of an unknown animal derived product sample based on specific panels of genes that provide a source for the generation of DNA methylation profiles which are specific for distinct suppliers who may or may not be certified suppliers. In particular, DNA methylation profiling may be used to determine the distinct supplier from which animal derived products originate from and/or if the animal-derived product has a distinct certification.

### BACKGROUND OF THE INVENTION

Sustainable food production is presently considered among the globally most important societal needs. As the value chains of the agriculture and aquaculture industries are highly complex, certificates have been established to reinforce consumer relationships and trust. However, certificates are based on audits at specific farms and can be easily tampered by moving livestock from non-certified farms to certified farms. Furthermore, surveillance of sustainable farming practices is spotty and largely limited to audits. As “bad” farming practices are widespread in the industry, there is an urgent need for a tampering-resistant certificate.

Further, there are people in the world that abide by dietary restrictions that relate specifically to a tradition or a religion that they follow. For example, some Jewish people eat only foods that have been deemed Kosher in accordance with Jewish law. Similarly, some people from the Muslim religion may desire to eat only foods that have been deemed Halal in accordance with Islamic law. Other people may have other dietary restrictions that relate to other traditions, religions, ethical codes, or the like. Both Kosher certification (i.e., designation of a food item as Kosher) and Halal certification (i.e., designation of a food item as Halal) of food, including meat involve the conformance with specific laws that relate to the slaughter of animals. For example, Judaic ritual slaughter (Shechita) and Islamic ritual slaughter (Zabihah) require an animal to be cut across the neck with a non-serrated blade in a single clean attempt to sever main blood vessels of the animal, and to drain the blood of the animal. Health, safety and also animal welfare considerations demand that the origins of animal products, and in particular meat products, should be traceable, so that quality assurance audits, and monitoring procedures can be effectively and reliably carried out. There is, however, no present means of accurately and reliably tracing the origins of meat. In particular, outside of trusting the labels found on the meat packets and shops, there is no reliable, effective and easy to use method for distinguishing halal meat from non-halal meat and kosher meat from non-kosher meat. That is to say, although meat may be certified as Kosher (i.e., allowed under the Jewish tradition) or Halal (i.e., allowed under the Islamic tradition), buyers may be unable to determine whether the meat was properly slaughtered based on visual inspection. Moreover, when consuming food at a restaurant, customers may be unable to view an inspection label affixed to packaging of meat prior to preparation of the meat. Furthermore, a customer may be unable to determine a type of animal from which the meat is derived based on visual inspection. This may result in a person who abides by a dietary restriction being hesitant to consume animal products.

Physical conditions and/or chemical conditions of an animal may be altered based on the way the animal has been slaughtered. For example, it is well known that cortisol levels of animals for which Kosher slaughter or Halal slaughter may be performed may be different from cortisol levels of animals that undergo another means of slaughter. Also, different types of meats obtained from different animals may be associated with physical differences and/or chemical differences. For example, proteins from pork meat of a pig may differ from proteins from beef meat of a cow. Differences may also exist in relation to different breeds of a particular type of animal, different cuts of meat of a particular animal, or the like. This may limit an effectiveness of comparing an unknown sample of meat to a set of known reference samples of meat.

Recent studies have shown that DNA methylation patterns of animals contain important information about their rearing conditions. For example, a comparison of genome-wide patterns of methylation and variation at the DNA level revealed that a highly significant proportion of epigenetic variation could be associated with fitness differences and rearing conditions such as captivity in salmon (Le Luyer J et al. 2017 PNAS vol 114, no 49).

Koop et al. and Rhein et al. also separately confirmed that methylation patterns are maintained postmortem and are consistent regardless of the level of decomposition of the sample. One of the only aspects that still affects the methylation pattern of the sample would be the DNA integrity and amount of DNA that is found in the sample (Koop et al., 2021 International Journal of Legal Medicine 135:167-173 and Rhein et al., 2015 Frontiers in Genetics 6:182).

In view of the above, there is an urgent need to provide means for identifying and quality controlling the certification of organisms, in particular food and more particularly animal material derived to satisfy the dietary restrictions relating to a tradition, a religion, an ethical code, or the like followed strictly by people.

### DESCRIPTION OF THE INVENTION

The present invention attempts to solve the problems above by providing a method using DNA methylation patterns to distinguish one type of animal derived product from another type of animal derived product to be able to determine the supplier and/or slaughterhouse and thereby certify the product accurately and reliably. The present invention is based on the finding that resilience to environmental exposures such as stress, climate, light, and diet and is a fundamental concept of biology and results in the adaptation of an organism to its environment. The capability to adapt to the environment and maintain the adapted biological pattern depends on epigenetic mechanisms, including DNA methylation. In particular, the present invention is based on the finding that the method of slaughtering an animal for food consumption may also result in changes in epigenetic mechanisms of the animal, including DNA methylation patterns.

The inventors have unexpectedly found that this property can be utilized to identify “epigenetic fingerprints” on the genome that are specific to the animal that has been slaughtered using a specific means. For example, these ‘epigenetic fingerprints’ may be specific for any product that stems from the very animal (an individual). Based on these findings, the present invention provides means to identify the specific method of slaughter of a particular animal, for example rearing animals also known as livestock and poultry from which an animal-derived product comes from. In particular, the method according to any aspect of the present invention may be used to trace the animal-derived product to the very source (individual animal, batch and/or slaughterhouse) from which the product originates. The method according to any aspect of the present invention may then be used to accurately and reliably determine the method of slaughter of an animal and then provide and/or confirm the certification of quality or certification of any sample that originates from the animal. Further, the method according to any aspect of the present invention may also be used to identify if any animal-derived product is accurately and reliably certified, particularly where the certification is based on the method of slaughter of the animal.

According to one aspect of the present invention, there is provided a method for certifying quality of an animal-derived product sample, the method comprising the steps of:
(a) determining a test methylation profile of one or more pre-selected methylation sites within the genomic material contained in a biological sample obtained from animal-derived product sample; and
(b) comparing the test methylation profile determined in (a) with one or more predetermined reference methylation profiles, wherein each of the predetermined reference methylation profiles is from a different animal and each of the different animals is from at least one certified supplier,
   wherein if the test methylation profile of (a) is significantly similar to one of the predetermined reference methylation profiles, the animal-derived product sample is confirmed from originating from the certified supplier from which the animal with the predetermined reference profile is obtained and the animal-derived product sample has the same certified quality as that of the animal.

According to another aspect of the present invention, there is provided a method of determining the supplier from which a test animal-derived product sample originates, the method comprising the steps of:
(a) determining a test methylation profile of one or more pre-selected methylation sites within the genomic material contained in the test animal-derived product sample; and
(b) comparing the test methylation profile determined in (a) with a panel of predetermined reference methylation profiles of the same biological taxon of the test animal from which the product sample derives, wherein each of the predetermined reference methylation profiles is from a different reference animal and/or different supplier,
   wherein if the test methylation profile of (a) is significantly similar to one of the predetermined reference methylation profiles, the test animal-derived product sample is confirmed of originating from a first supplier from which a first reference animal with the predetermined reference profile is obtained.

The method according to any aspect of the present invention may thus enable the tracing of the original slaughterhouse, farm, produce, supplier etc. from which the test animal material originates from and thereby the test animal derived product.

As used herein, the term ‘animal-derived product’ refers to products that originate from animals. In particular, the term ‘test animal-derived product’ refers to the sample or subject in question that is to undergo the method according to any aspect of the present invention. These products from animals may include meat and meat products, also including fat, flesh, blood, and lesser-known products, such as isinglass and rennet, poultry products (meat and eggs), dairy products (milk and cheese), and non-food products such as fiber (wool, mohair, cashmere, leather and the like). Animal-derived products may also include products that can be made using animal products (e.g. fat) such as soap, creams and such. In one example, the animal-derived product is meat, eggs, blood, brain, sperm, milk and any other tissue or sample that provides genomic DNA. In particular, the animal-derived product is meat.

The term ‘certification of quality’ refers to a certificate or a confirmation given by designated certification agencies that endorse the quality of a particular animal derived product, including food for use and/or consumption by human beings. The term ‘certification of quality’ is used interchangeably with the term ‘certification’. In particular, certification of quality does not necessarily refer specifically to meat quality aspects such as taste, texture, colour, moisture etc. These certifications are usually found on the packaging of the animal-derived product including food to be consumed and are printed by the manufactures of the products. Examples of certifications of distinct food quality may include ‘Haltungsform’, ‘Tierwohl’, ‘Ohne Gentechnik’, ‘halal’, ‘kosher’, and other safe labels that confirm that a product sold has been prepared in accordance with specific religious or safety regulations. Specifically, the term ‘certification of food quality’ refers to a certificate or a confirmation given by designated certification agencies that endorse the quality, source or means of slaughter of a particular food for consumption by human beings. According to any aspect of the present invention, the certified quality may be a distinct certified food quality, or distinct certification and this may be kosher, non-kosher, halal or non-halal.

In one example, the distinct certified food quality or certification of the sample X according to any aspect of the present invention may be kosher, non-kosher, halal or non-halal. More in particular, kosher or halal refers to the sample X originating from an animal that was slaughtered by a single cut across the throat severing both carotid arteries, both jugular veins, both vagus nerves, the trachea and/or the esophagus. Even more in particular, the animal is drained of blood.

The term ‘kosher’ used in combination with food according to any aspect of the present invention refers to food that conforms to Jewish dietary regulations of kashrut (dietary law) or food that may be consumed according to halakha (law). Kosher used in relation to meat relates particularly to a manner in which animals are prepared for consumption. According to Jewish tradition, meat may be considered kosher when the meat comes from animals that have been slaughtered according to Jewish law where the animal is killed by a single cut across the throat to a precise depth, severing both carotid arteries, both jugular veins, both vagus nerves, the trachea and the esophagus, no higher than the epiglottis and no lower than where cilia begin inside the trachea, causing the animal to bleed to death. Such slaughter is to be carried out using a large, razor-sharp knife, which is checked before each slaughter to ensure that it has no irregularities (such as nicks and dents). The slaughter is usually also carried out by a shochet or a rabbi. Kosher meat usually refers to most meats excluding pig. In particular, kosher meat may be selected from beef, chicken, lamb, mutton, goat meat and mixtures thereof. Kosher meat does not include shellfish, which under Jewish tradition is not permitted for consumption. Any food or meat that does not fall within the definition of ‘kosher’ will then be considered as ‘non-kosher’.

The term ‘halal’ used in combination with food according to any aspect of the present invention refers to food that conforms to Islamic dietary laws and especially meat processed and prepared in accordance with those requirements. Similar to the way kosher meat is prepared, in Islamic tradition, animals are slaughtered according to Dhabihah where the animal is slaughtered using a cut across the neck with a non-serrated sharp blade in a single clean attempt to make an incision that cuts the front of the throat, oesophagus and jugular veins but not the spinal cord. In addition to the direction, permitted animals should be slaughtered upon utterance of the Islamic prayer Bismillah. The animal must also be drained of blood after the slaughter. The slaughter must be performed by an adult Muslim. Halal meat usually refers to most meats excluding pig. In particular, halal meat may be selected from beef, chicken, lamb, mutton, goat meat and mixtures thereof. Although Islamic traditions permit consumption of shellfish, since there is no special method of preparing shellfish, all shellfish may be considered halal. Any food or meat that does not fall within the definition of ‘halal’ will then be considered as ‘non-halal. The definition of halal is further provided in https://www.smiic.org/en/project/24 (Organisation of Islamic Cooperation (OIC)/Standards and Metrology Institute for the Islamic Countries (SMIIC), OIC/SMIIC 1:2019 General Requirements for Halal Food. accessed on 8 Jun. 2022)

The term “test” used in conjunction with the term subject and/or animal in the present disclosure refers to an entity that is subjected to the method according to any aspect of the present invention and is the basis for an analysis application of the present invention. An “(individual) test subject”, an “(individual) group of test subjects” or a “test profile” or an ‘test animal derived product’ is therefore a (individual) subject or group of subjects being tested according to the invention or a profile being obtained or generated in this context. Conversely, the term “reference” shall denote, mostly predetermined, entities which are used for a comparison with the test entity. For example, the term ‘reference animal’ refers to an animal used for comparison or as a control in reference to the ‘test animal’. Similarly, the term ‘sample’ and/or ‘test animal-derived product sample’ used in accordance with any aspect of the present invention refers to an entity that may be subject to the method of the present invention. In particular, a sample may be any (test) animal-derived product that may be subject to the method of the present invention to determine the distinct certified quality by first determining the source or supplier (i.e. slaughterhouse or milk or chicken farm or the like) from which the test animal-derived product is obtained from. For example, an animal-derived product sample may be a piece of meat tested according to any aspect of the present invention to determine the supplier or slaughterhouse from which the meat originates. This information may then be able to trace the meat back to the slaughterhouse and the accuracy of the certification of the meat sample (i.e. halal, non-halal, kosher or non-kosher, organic or non-organic and the like) may be confirmed. In another example, a (test) animal-derived product sample may be another piece of meat which is tested according to any aspect of the present invention to determine the actual slaughterhouse from which the meat originates and thereby enabling to meat to be accurately certified based on the certification the slaughterhouse has been given. The method according to any aspect of the present invention therefore enables the meat to be traced back to its source (i.e. slaughterhouse, supplier, batch and/or animal). Blockchain may also be used to make the information easily available for the consumer.

The method according to any aspect of the present invention may be used to identify an unknown sample (i.e. animal-derived product sample) based on DNA methylation patterns. These DNA methylation patterns may then be compared with reference DNA methylation patterns to trace the animal-derived product sample back to the slaughterhouse or farmhouse from which the sample originates and then determine whether the unknown sample corresponds to an animal which has been slaughtered according to Kosher, Halal, non-Kosher or non-Halal practices. In this way, a buyer or a consumer of meat can verify that meat being sold as or marketed as being Kosher or Halal is genuine.

The term ‘supplier’ used herein refers to the entity that provides the animal derived product. This can be considered the slaughterhouse according to any aspect of the present invention as the slaughterhouse is where the animal is killed according to specific practices and the different animal derived products which include animal products and animal by-products originate from. The slaughterhouse or slaughter facility typically slaughters the animal and then chills, ages and cuts the carcass into the various cuts of meat and packs those cuts for shipment to distributors and retailers. The slaughterhouse is also where non-food products originate from. The supplier may be certified. A ‘certified supplier’ means a supplier that has been assessed for quality, business, technical, environmental, health and safety considerations and subsequently approved by a government or third-party agency or agencies. In one example, the supplier may be certified ‘halal’. This means that all the animal derived products that are supplied by the certified supplier will be certified ‘halal’. In another example, the supplier may be certified ‘kosher’. This means that all the animal derived products that are supplied by or originated from the certified supplier will be certified ‘kosher’. The supplier is certified kosher or halal where the animal is slaughtered by a single cut across the throat severing both carotid arteries, both jugular veins, both vagus nerves, the trachea and/or the esophagus. In addition, or alternatively, the supplier is certified kosher or halal where the animal is drained of blood. The animal may be selected from the group consisting of cow, sheep, goat, camel, chicken, goose, duck, and turkey.

As used herein, the term “comprising” is to be construed as encompassing both “including” and “consisting of”, both meanings being specifically intended, and hence individually disclosed aspects of the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g., “a”, “an” or “the”, this includes a plural of that noun unless something else is specifically stated.

In context of the present invention, the terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “methylation status” refers to the status of a specific methylation site (i.e. methylated vs. non-methylated) which means a residue or methylation site is methylated or not methylated. Then, based on the methylation status of one or more methylation sites, a methylation profile may be determined. Accordingly, the term “methylation profile” or also “methylation pattern” refers to the relative or absolute concentration of methylated C residues or unmethylated C residues at any particular stretch of residues in the genomic material of a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., the DNA from a sample nucleic acid from a test subject) are methylated as compared to another sequence from a different region or from a different individual (e.g., relative to normal nucleic acid or to the standard nucleic acid of the reference sequence), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG- and CpNpN-sequences may be considered the most common. In principle, other naturally occurring nucleotides may also be methylated but they will not be taken into consideration with regard to any aspect of the present invention.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro.

As used herein, a “methylated nucleic acid molecule” refers to a nucleic acid molecule that contains one or more nucleotides that is/are methylated.

The term “bisulfite” as used herein encompasses any suitable type of bisulfite, such as sodium bisulfite, or another chemical agent that is capable of chemically converting a cytosine (C) to a uracil (U) without chemically modifying a methylated cytosine and therefore can be used to differentially modify a DNA sequence based on the methylation status of the DNA, e.g., U.S. Pat. Pub. US 2010/0112595 (Menchen et al.). As used herein, a reagent that “differentially modifies” methylated or non-methylated DNA encompasses any reagent that modifies methylated and/or unmethylated DNA in a process through which distinguishable products result from methylated and non-methylated DNA, thereby allowing the identification of the DNA methylation status. Such processes may include, but are not limited to, chemical reactions (such as a C to U conversion by bisulfite) and enzymatic treatment (such as cleavage by a methylation-dependent endonuclease). Thus, an enzyme that preferentially cleaves or digests methylated DNA is one capable of cleaving or digesting a DNA molecule at a much higher efficiency when the DNA is methylated, whereas an enzyme that preferentially cleaves or digests unmethylated DNA exhibits a significantly higher efficiency when the DNA is not methylated.

In context of the present invention also any “non-bisulfite-based method” and “non-bisulfite-based quantitative method” are comprised to test for a methylation status at any given methylation site to be tested. Such terms refer to any method for quantifying methylated or non-methylated nucleic acid that does not require the use of bisulfite. The terms also refer to methods for preparing a nucleic acid to be quantified that do not require bisulfite treatment. Examples of non-bisulfite-based methods include, but are not limited to, methods for digesting nucleic acid using one or more methylation sensitive enzymes and methods for separating nucleic acid using agents that bind nucleic acid based on methylation status. The terms “methyl-sensitive enzymes” and “methylation sensitive restriction enzymes” are DNA restriction endonucleases that are dependent on the methylation state of their DNA recognition site for activity. For example, there are methyl-sensitive enzymes that cleave or digest at their DNA recognition sequence only if it is not methylated. Thus, an unmethylated DNA sample will be cut into smaller fragments than a methylated DNA sample. Similarly, a hypermethylated DNA sample will not be cleaved. In contrast, there are methyl-sensitive enzymes that cleave at their DNA recognition sequence only if it is methylated. As used herein, the terms “cleave”, “cut” and “digest” are used interchangeably.

A “biological sample” in context of the invention may comprise any biological material obtained from the subject or group of subjects that contains genomic material, and may be liquid, solid or both, may be tissue or bone, or a body fluid such as blood, lymph, etc. In particular the biological sample useful for the present invention may comprise biological cells or fragments thereof.

As used herein, the term “pre-selected methylation sites” refers to methylation sites that were selected from genes or regions that showed the highest degree of methylation variation during the training of the method and fulfils certain quality criteria such as a minimum sequencing coverage of ≥5× were considered and for ≥5 qualified CpG sites. Additionally, genes that have an average methylation level <0.1 or an average methylation level >0.9 can be excluded due to their limited dynamic range. “Reference methylation profiles” may be defined on the basis of multiple training samples using multivariate statistical methods, such as such as Principal Component analysis or Multi-Dimensional Scaling.

The term “pre-determined reference profile” used in the context of the present invention refers to a typical or standard methylation profile of the genomic material of a type of reference animal-derived product from a distinct or specific supplier and/or with a specific certification of quality. The pre-determined reference profile may be obtained from a control subject. For example, the control may be a piece of meat from an animal that has been slaughtered by a certified supplier. A panel of pre-determined reference profiles may be prepared for each certified supplier where each pre-determined profile is unique for each animal that has been slaughtered by the certified supplier and/or each part of the animal (i.e. tissue, muscle, blood, skin) has its own unique pre-determined methylation reference profile that also forms a part of the panel of pre-determined reference profiles. Different certified suppliers may then have different panels of pre-determined reference profiles. For example, each panel may be specific for a single animal where each reference profile may be distinct for a part of the animal from which the genomic material is extracted. When a test methylation profile from an unknown animal-derived product sample is obtained, this is then compared with the different panels of pre-determined reference profiles to trace the actual animal from which the animal-derived product sample comes from. If the test methylation profile is significantly similar to one of the pre-determined reference profiles, then the origin of the animal-derived product sample can be determined, and the appropriate certification may be given to the sample, or the sample can be certified for its quality. If the test methylation profile is found to be not significantly similar to any one of the pre-determined reference profiles, then the animal-derived product sample is confirmed not to be from the list of certified suppliers and cannot be given the certification or cannot be certified for its quality.

For example, the control may a type of meat of the same species or the same animal taxon as the animal-derived product sample or unknown sample which has a known certification or certification of food quality. Alternatively, the pre-determined reference profile may be obtained from different types of meat with a known certification. In one example, the control may be chicken meat that is certified ‘halal’ from a first certified supplier. The halal chicken meat may be a mix of different parts of the chicken (i.e., breast, thigh, kidney, liver etc.) from a single chicken. In another example, the halal chicken meat may be a mix of different parts of the chicken (i.e., breast, thigh, kidney, liver etc.) from a few chickens from a first certified supplier. In yet another example, the halal chicken meat may be from one part of a single chicken from a first certified supplier. From the halal chicken meat, a methylation profile may be obtained to be the pre-determined reference profile (the control) that may be used to determine if the test sample (meat) is halal or non-halal by determining if the test sample is from the first certified supplier. The methylation profile of different types of halal meat from one species of animal may be identical. The methylation profile of different types of halal meat from different species of animals may also be identical. In another example, the control may be chicken meat that is certified ‘kosher. The kosher chicken meat may be a mix of different parts of the chicken (i.e. breast, thigh, kidney, liver etc.) from a single chicken from a second certified supplier. In another example, the kosher chicken meat may be a mix of different parts of the chicken (i.e., breast, thigh, kidney, liver etc.) from a few chickens from the second certified supplier. In yet another example, the kosher chicken meat may be from one part of a single chicken from the second certified supplier. From the kosher chicken meat, a methylation profile may be obtained to be the pre-determined reference profile (the control) that may be used to determine if the test sample (meat) is kosher or non-kosher by first determining if the test sample is from the second certified supplier. The methylation profile of different types of kosher meat from one species of animal may be identical. The methylation profile of different types of kosher meat from different species of animals may also be identical.

There may be a compilation of several pre-determined reference profiles and comparing the methylation profile of the test sample with the pre-determined reference profiles in the compilation may enable identifying the specific pre-determined reference profile that is (significantly) similar to the methylation profile of the test sample and then the test sample may be traced back to its origins or to the supplier and thereby the certification of quality of the test sample may be deduced to be that of the pre-determined reference profile. In one example, the pre-determined reference profiles may include methylation profiles of different parts of meat (i.e., breast, thigh, kidney, liver, shoulder, ribs, intestines, etc.) from a single animal (chicken, goat, cow, lamb, sheep etc.) or at least two animals of the same species or at least two animals from different species. The different unique methylation profiles are reference epigenetic signatures of meat from a specific source (EpiTrace®).

In particular, the panel of predetermined reference methylation profiles according to any aspect of the present invention is distinct for different test animal-derived products. That is to say, each predetermined reference methylation profile is distinct for a single animal-derived product from one animal from one unique supplier. The panel of predetermined reference methylation profiles may thus include many different predetermined reference methylation profiles from different parts of a single animal from the unique supplier, many different predetermined reference methylation profiles from different animals from the unique supplier and many different predetermined reference methylation profiles from different parts of different animals from different suppliers. There will also be different panels of predetermined reference methylation profiles for different animal taxon, and the relevant panel of predetermined reference methylation profiles unique for an animal taxon will depend on the animal taxon of the test animal.

In particular, the first supplier according to any aspect of the present invention is certified, and the test animal-derived product sample has the same certification as the first reference animal from which the significantly similar predetermined reference profile is obtained.

More in particular, the first supplier according to any aspect of the present invention is certified kosher or halal where the animal is slaughtered by a single cut across the throat severing both carotid arteries, both jugular veins, both vagus nerves, the trachea and/or the esophagus. In addition, or alternatively, the first supplier may be certified kosher or halal where the animal is drained of blood.

The term “significantly similar” in context of the present disclosure, and in particular in context with the comparison of methylation profiles (such as the comparison between test profiles (from test subject(s) (i.e. sample X) and reference profiles) shall mean a similarity observed by statistical means (i.e. by using bioinformatics) and/or also by observation using the eye. A significant similarity is observed for example if a test profile overlaps with a reference profile that is defined by multiple training samples through multivariate statistical methods, such as Principal Component analysis or Multi-Dimensional Scaling. In particular, a test profile is significantly similar to the pre-determined reference profile if more than 50, 55, 60, 65, 70, 75, 80, 85, 90, 95, 96, 97, 98, 99% of the methylation pattern/profile overlaps with that of the reference profile. A similarity of a test profile to more than one, such as two, three or even all reference profile reduces the significance of the similarity. Of course, the similarity of the test methylation profile to the reference methylation profile takes into consideration the experimental error that occurs in all methods.

The animal-derived product sample may be a single type of meat, different types of meat, a single part of a type of meat, different parts of a single type of meat or different parts of different types of meat. The sample may be from any biological entity having a DNA genome and DNA genome methylation. In particular, the methylation site is a CpG site. The biological entity may be any animal excluding a pig. In particular, the animal may be selected from the group consisting of chicken, lamb, camel, cow, goat, sheep, horse, donkey, turkey, duck, goose, quail, rabbit and mule. More in particular, the animal may be selected from the group consisting of cow, sheep, goat, camel, chicken, goose, duck and turkey. The term ‘meat’ herein may thus be understood to include chicken, lamb, beef, mutton, goat, camel, chicken, goose, duck, turkey and mixtures thereof.

The one or more pre-selected methylation sites in (a) are methylation sites associated with tissue specific gene expression, preferably wherein the pre-selected methylation sites are associated with gene expression of one distinct tissue.

The tissue may be selected from
(i) metabolic tissue such as gut tissue, said gut tissue preferably being ileum or jejunum,
(ii) muscular tissue,
(iii) skin tissue, and
(iv) organ tissue, said organ tissue preferably being hepatic and/or pancreatic tissue.

According to a further aspect of the present invention, there is provided a method of verifying certification of a test animal-derived product sample, the method comprising the steps of:
(a) determining a test methylation profile of one or more pre-selected methylation sites within the genomic material contained in the test animal-derived product sample; and
(b) comparing the test methylation profile determined in (a) with a panel of predetermined reference methylation profiles of the same biological taxon of the test animal from which the product sample derives, wherein each of the predetermined reference methylation profiles is from a different reference animal and/or different certified supplier,
   wherein if the test methylation profile of (a) is significantly similar to one of the predetermined reference methylation profiles, the test animal-derived product sample is confirmed of originating from the certified supplier from which the reference animal with the predetermined reference profile is obtained and if the test methylation profile of (a) is not similar to any one of the predetermined reference methylation profiles, the test animal-derived product sample is confirmed of not originating from a certified supplier.

In particular, the certified supplier is certified kosher or halal where the animal is slaughtered by a cut across the throat severing both carotid arteries, both jugular veins, both vagus nerves, the trachea and/or the esophagus. Additionally, or alternatively, the certified supplier is certified kosher or halal where the animal is drained of blood.

According to yet a further aspect of the present invention, there is provided a certification of a test animal derived product sample certifying that the test animal was slaughtered by a single cut across the throat severing both carotid arteries, both jugular veins, both vagus nerves, the trachea and/or the esophagus,
wherein the method for determining that the test animal was slaughtered by a single cut across the throat severing both carotid arteries, both jugular veins, both vagus nerves, the trachea and/or the esophagus comprises the steps of:
(a) determining a test methylation profile of one or more pre-selected methylation sites within the genomic material contained in the test animal-derived product sample; and
(b) comparing the test methylation profile determined in (a) with a panel of predetermined reference methylation profiles of the same biological taxon of the test animal from which the product sample derives, wherein each of the predetermined reference methylation profiles is from a different reference animal and/or different supplier,
   wherein if the test methylation profile of (a) is significantly similar to one of the predetermined reference methylation profiles, the test animal was slaughtered by a single cut across the throat severing both carotid arteries, both jugular veins, both vagus nerves, the trachea and/or the esophagus. Additionally, or alternatively, the test animal is drained of blood.

According to another aspect of the present invention, there is provided a method for determining a certification of quality of an animal derived product sample, the method comprising:

comparison of a test methylation profile obtained from genomic material of the animal derived product sample with one or more predetermined reference methylation profiles each being specific for a distinct certified quality wherein the method comprises the steps of:
a) determining the test methylation profile of one or more pre-selected methylation sites within the genomic material obtained from the animal derived product sample; and
b) comparing the test methylation profile determined in (a) with one or more predetermined reference methylation profiles, wherein each of the one or more predetermined reference methylation profiles is specific for a distinct certification or certified quality based on the certification of the supplier;
wherein if the test methylation profile is significantly similar to one of the predetermined reference methylation profiles, the animal derived product sample is traced back to the supplier and has the same certification or distinct certified quality as that of the predetermined reference methylation profile or supplier.

Every aspect of the present invention is based on the surprising identification of methylation profiles in a subset of genes of a sample, in particular meat, which are characteristic for a specific certification or certification of food quality or specific to an animal from a unique certified supplier. Different samples with different certifications or from different animals from different certified suppliers have different methylation profiles in the same subset of genes.

The distinct certified quality or certification of the animal derived product sample may be kosher, non-kosher, halal or non-halal. In particular, the animal derived product sample is meat.

According to another aspect of the present invention, there is provided a method for confirming or rejecting an assumed certification or certification of quality of an animal derived product sample, the method comprising
comparison of a test methylation profile obtained from genomic material of the animal derived product sample with one or more predetermined reference methylation profiles each being specific for an animal from a unique certified supplier.

According to a further aspect of the present invention, there is provided a method for developing a test system for confirming an assumed certification of an unknown sample, the method comprising the steps of:
a. determining the methylation status of one or more methylation sites within genomic material obtained from the unknown sample;
b. selecting from the one or more methylation sites a reference panel of methylation sites which is characterized by a specific and distinct differential methylation profile for each certified supplier;
c. obtaining a test system by assigning a reference methylation profile for each of the known certified suppliers; and
wherein a comparison of a test methylation profile obtained from the sample with the reference methylation profiles obtained in (c) allows for confirming the assumed certification of the sample.

According to yet another aspect of the present invention, there is provided a use of DNA methylation profiling for determining certification or certified quality of an animal-derived product sample. The comparison of a test methylation profile obtained from genomic material of the sample with one or more predetermined reference methylation profiles each being specific for a distinct certified supplier, determines the source of the product and thereby the certification or certified quality of the sample. In particular, the distinct certified quality of the sample is kosher, non-kosher, halal or non-halal.

### BRIEF DESCRIPTIONS OF FIGURES

FIG. 1 is a flow diagram of the method of the present invention for certifying the food quality of a sample

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Certification of Food as Halal Using DNA Methylation

An Islamic religious leader or certifying body will certify a meat slaughtering facility to confirm that it complies with Islamic requirements and the meat is being slaughtered according to Islamic practices declaring that the meat deriving from this facility is halal.

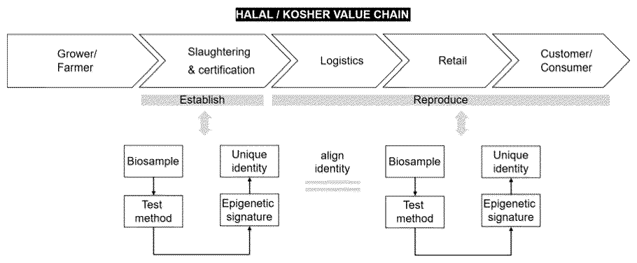

Halal chicken meat from a slaughtered batch of meat is then obtained from this facility and the sample is transported to a laboratory. DNA is extracted from the chicken meat. The extracted DNA is used for establishing the unique reference epigenetic signature of the meat batch (EpiTrace). The unique epigenetic signature is then assigned to the specific facility. The meat is then passed along the value chain as shown in FIG. 1.

A meat sample (X) is taken at any position in the value chain, e.g. at the supermarket or at a meat processor that is processing halal meat and shipped to the laboratory. This meat sample (X) is used to extract DNA. The extracted DNA is then used to establish a specific epigenetic signature. If both epigenetic signatures are from the identical batch of slaughtered meat, they will be identical, and origin of the halal meat is verified and can be traced back to the location which conducted the halal slaughter practice. Because incoming meat may derive from different and varying locations, each meat batch requires the assignment of an epigenetic signature.

1. A method of determining the supplier from which a test animal-derived product sample originates, the method comprising the steps of:
(a) determining a test methylation profile of one or more pre-selected methylation sites within the genomic material contained in the test animal-derived product sample; and
(b) comparing the test methylation profile determined in (a) with a panel of predetermined reference methylation profiles of the same biological taxon of the test animal from which the product sample derives, wherein each of the predetermined reference methylation profiles is from a different reference animal and/or different supplier,
wherein if the test methylation profile of (a) is significantly similar to one of the predetermined reference methylation profiles, the test animal-derived product sample is confirmed of originating from a first supplier from which a first reference animal with the predetermined reference profile is obtained.

2. The method according to claim 1, wherein the animal is selected from the group consisting of chicken, lamb, cow, goat, sheep, camel, horse, donkey, turkey, duck, goose, quail, rabbit and mule.

3. The method according to claim 1, wherein the panel of predetermined reference methylation profiles is distinct for different test animal-derived products.

4. The method according to claim 1, wherein the first supplier is certified, and the test animal-derived product sample has the same certification as the first reference animal from which the significantly similar predetermined reference profile is obtained.

5. The method according to claim 1, wherein the first supplier is certified kosher or halal where the animal is slaughtered by a single cut across the throat severing both carotid arteries, both jugular veins, both vagus nerves, trachea and/or esophagus.

6. The method according to claim 4, wherein the first supplier is certified kosher or halal where the animal is drained of blood.

7. The method according to claim 5, wherein the animal is selected from the group consisting of cow, sheep, goat, camel, chicken, goose, duck and turkey.

8. The method according to claim 1, wherein the one or more pre-selected methylation sites in (a) are methylation sites associated with tissue specific gene expression, preferably wherein the pre-selected methylation sites are associated with gene expression of one distinct tissue.

9. A method of verifying certification of a test animal-derived product sample, the method comprising the steps of:
(a) determining a test methylation profile of one or more pre-selected methylation sites within the genomic material contained in the test animal-derived product sample; and
(b) comparing the test methylation profile determined in (a) with a panel of predetermined reference methylation profiles of the same biological taxon of the test animal from which the product sample derives, wherein each of the predetermined reference methylation profiles is from a different reference animal and/or different certified supplier,
wherein if the test methylation profile of (a) is significantly similar to one of the predetermined reference methylation profiles, the test animal-derived product sample is confirmed of originating from the certified supplier from which the reference animal with the predetermined reference profile is obtained and if the test methylation profile of (a) is not similar to any one of the predetermined reference methylation profiles, the test animal-derived product sample is confirmed of not originating from a certified supplier.

10. The method according to claim 9, wherein the certified supplier is certified kosher or halal where the animal is slaughtered by a single cut across the throat severing both carotid arteries, both jugular veins, both vagus nerves, trachea and/or esophagus.

11. The method according to claim 9, wherein the certified supplier is certified kosher or halal where the animal is drained of blood.

12. Certification of a test animal derived product sample certifying that the test animal was slaughtered by a single cut across the throat severing both carotid arteries, both jugular veins, both vagus nerves, the trachea and/or the esophagus,
wherein the method for determining that the test animal was slaughtered by a single cut across the throat severing both carotid arteries, both jugular veins, both vagus nerves, the trachea and/or the esophagus comprises the steps of:
(a) determining a test methylation profile of one or more pre-selected methylation sites within the genomic material contained in the test animal-derived product sample; and
(b) comparing the test methylation profile determined in (a) with a panel of predetermined reference methylation profiles of the same biological taxon of the test animal from which the product sample derives, wherein each of the predetermined reference methylation profiles is from a different reference animal and/or different supplier,
wherein if the test methylation profile of (a) is significantly similar to one of the predetermined reference methylation profiles, the test animal was slaughtered by a single cut across the throat severing both carotid arteries, both jugular veins, both vagus nerves, trachea and/or esophagus.

13. The method according to claim 12, wherein the test animal is drained of blood.

14. The method according to claim 12, wherein the animal is selected from the group consisting of cow, sheep, goat, camel, chicken, goose, duck and turkey.

15. The method according to claim 1, wherein the test animal-derived product sample is meat, eggs, milk and any other tissue or sample that provides genomic DNA.
